(19) United States
(12) Patent Application Publication
(10) Pub. No.: US 2026/0007377 A1
(43) Pub. Date: Jan. 8, 2026

(54) DEVICE TO FACILITATE SAFE TRANSPORT OF MOBILE ADAPTATION KIT

(71) Applicant: Jubilant Draximage Inc., Kirkland (CA)

(72) Inventors: Hesam Nasery, Kirkland (CA); Emilia Benevento, Kirkland (CA); Veronique Audet, Kirkland (CA); Sara Devlin, Kirkland (CA); Giuseppe Carlucci, Kirkland (CA); Kasturi Sinha Roy, Greater Noida (IN); Dinesh Kumar Sarwal, Greater Noida (IN)

(21) Appl. No.: 19/329,271

(22) Filed: Sep. 15, 2025

Related U.S. Application Data
(63) Continuation-in-part of application No. 18/182,430, filed on Jun. 12, 2023, now Pat. No. 12,417,858.
(60) Provisional application No. 63/319,048, filed on Mar. 11, 2022.

(51) Int. Cl. A61B 6/00

(52) U.S. Cl. CPC A61B 6/4405; A61B 6/547

(57) ABSTRACT

The present invention relates to a medical radioisotope transport system that securely holds the radiopharmaceutical imaging system or medical elution system to the floor of a vehicle and ensures the protection of the elution system during transportation. More particularly, the present invention relates to a mobile adaptation kit, comprising a floor frame assembly installed in a transport vehicle configured to support a mobile medical radioisotope elution system; and a T-bar that slides inside and out of the floor frame assembly, wherein the T-bar comprises a gasket and nylon sheets. The floor frame assembly comprises a floor frame that is installed and remains within the vehicle or van; a floor frame cover; an angled rail; a pivoting piece; a quick-release pin with a high-capacity pin that locks one end of the T-bar in place with the floor frame; and a locking mechanism, in order to safely and securely transport radiopharmaceuticals and medical imaging system, ensuring compliance to the regulatory and quality requirements. The transport system is easy to use and addresses the complexities involved in prior available methods for transportation of radiopharmaceutical imaging systems or medical radioisotope elution systems.

### TECHNICAL FIELD OF THE INVENTION

The present invention relates in general to a medical radioisotope transportation device and mechanism with enhanced safety features and a process implementing the same. More particularly, the present invention relates to a mobile adaptation kit, comprising a floor frame assembly installed in a transport vehicle configured to support a mobile medical radioisotope elution system; and a T-bar that slides inside and out of the floor frame assembly, in order to safely and securely transport radiopharmaceuticals and medical imaging system, ensuring compliance to the regulatory and quality requirements. A transport system as per the present invention is easy to use, and successfully overcomes the complexities involved in prior available methods for transportation of radiopharmaceutical imaging systems or medical radioisotope elution systems.

### BACKGROUND OF THE INVENTION

Radioisotopes play a pivotal role in the diagnosis and mitigation of various disease conditions. For example, ⁶⁰Co in treatment of cancer, ¹³¹I in treatment of hyperthyroidism, ¹⁴C in breath tests, ^{99m}Tc, and ⁸²Rb as tracers in myocardial perfusion imaging. Rubidium (⁸²Rb) is one such compound used as a positron emission tomography (PET) tracer for the non-invasive measurement of myocardial perfusion. The half-lives of some radiopharmaceuticals are very short. Thus, the whole imaging and administration procedure effectively needs to be completed within a very short time period. Due to these challenges, radiopharmaceuticals are usually prepared at on-site facilities having a suitable driving distance from the patient site to prevent undue decay of the radiopharmaceutical prior to use. Moreover, radiopharmaceutical systems carry radiation hazards hence safe transportation is essentially required to avoid any safety hazards.

Regulatory authorities such as USFDA (United States Food and Drug Administration) have set stringent rules and regulations for the safe transportation of radiopharmaceuticals due to harmful effects involved with undesirable and hazardous exposure to radioactive materials. However, due to the complexities involved and stringent regulatory requirements, the presently available tools are not cost-effective and complex in nature.

Hence, there exists an unmet urgent need for an advanced, simple, and cost-effective medical radioisotope transportation device and mechanism with enhanced safety features and without requiring a large capital investment.

The transport system as per the present invention fulfills all parameters established by regulatory authorities such as USFDA. The transport system as per the present invention fulfills an unmet need by providing easy and safe access of imaging facilities to patients, hospitals, and radio pharmacies without large capital investment. The transport system as per the present invention securely holds the medical radioisotope elution system to the floor of a vehicle and ensures protection from any undesirable damage to the medical radioisotope elution system during transportation without changing any quality parameters of the elution system. The transport system as per the present invention is used to securely lock the elution system in a vehicle such as a truck, van or trailer and can also be used to load or unload the elution system from the vehicle based on the need of the user or patient site. The transport system according to the present invention is a mobile flexdock floor mounted adaptation kit, which is an accessory to an elution system or a radiopharmaceutical generator system. It is a mechanical assembly that is compliant to applicable quality control testing at the manufacturing level for each of its components.

### SUMMARY OF THE INVENTION

The present invention aims to provide a medical radioisotope transportation device and mechanism with enhanced safety features and a process for implementing such system.

It is an object of the present disclosure to enhance the safety of the radiopharmaceutical systems during transportation.

It is an object of the present invention to provide a medical radioisotope transport system.

It is an object of the present disclosure to provide a medical radioisotope transport system that securely holds the mobile medical radioisotope elution system to the floor of a vehicle and ensures the protection of the elution system during transportation and use.

It is also an object of the present disclosure to provide a process for implementing the medical radioisotope transport system as per the present invention to the floor of a vehicle to ensure the protection of the mobile medical radioisotope elution system during transportation and use.

It is also an object of the present disclosure to provide a mobile adaptation kit, comprising:
a) a floor frame assembly installed in a transport vehicle configured to support a mobile medical radioisotope elution system; and
b) a T-bar that slides inside and out of the floor frame assembly, wherein the T-bar comprises a gasket and nylon sheets;
wherein the floor frame assembly comprises:
   i) a floor frame that is attached to the vehicle floor;
   ii) a floor frame cover with a top portion that creates the slot to install the T-bar inside and out of the floor frame assembly;
   iii) an angled rail positioned on each side of the floor frame assembly that installs under the floor frame cover;
   iv) a pivoting piece at the centre of the floor frame assembly;
   v) a quick-release pin with a high-capacity pin that locks one end of the T-bar in place with the floor frame; and
   vi) a locking mechanism that locks at one end of the T-Bar in position, in order to prevent sliding out of the radioisotope elution system;
wherein the gasket helps to absorb vibration during transportation; and the sheets help to reduce friction when inserting into the floor frame assembly.

It is also an object of the present disclosure to provide a mobile adaptation kit, comprising:
a) a floor frame assembly installed in a transport vehicle configured to support a mobile medical radioisotope elution system; and
b) a T-bar that slides inside and out of the floor frame assembly, wherein the T-bar comprises a gasket and nylon sheets;
wherein the floor frame assembly comprises:
   i) a floor frame that is attached to the vehicle floor;
   ii) a floor frame cover with a top portion that creates the slot to install the T-bar inside and out of the floor frame assembly;
   iii) an angled rail positioned on each side of the floor frame assembly that installs under the floor frame cover;
   iv) a pivoting piece at the centre of the floor frame assembly, installed with high load compression springs;
   v) a quick-release pin with a high-capacity pin that locks one end of the T-bar in place with the floor frame; and
   vi) a locking mechanism that locks at one end of the T-Bar in position, in order to prevent sliding out of the radioisotope elution system;
wherein the gasket helps to absorb vibration during transportation; and the nylon sheets help to reduce friction when inserting into the floor frame assembly; and
wherein the said mobile adaptation kit securely holds the medical radioisotope elution system to the floor of a vehicle and ensures protection from any undesirable damage to the medical radioisotope elution system during transportation.

It is also an object of the present disclosure to provide a mobile adaptation kit, comprising:
a) a floor frame assembly installed in a transport vehicle configured to support a mobile medical radioisotope elution system; and
b) a T-bar that slides inside and out of the floor frame assembly, wherein the T-bar comprises a gasket and nylon sheets; and wherein the gasket helps to absorb vibration during transportation; and the nylon sheets help to reduce friction when inserting into the floor frame assembly;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail positioned on each side of the floor frame assembly that installs under the floor frame cover, wherein the angled rail is used as a guide to align the T-bar into position;
   iv) a pivoting piece at the centre of the floor frame assembly, installed with high load compression springs on one end, wherein the pivoting piece gets activated when the T-bar is in the final position;
   v) a quick-release pin with a high-capacity pin that locks one end of the T-bar in place with the floor frame;
   vi) a sliding bolt latch that locks other end of the T-Bar in position, in order to prevent sliding out of the radioisotope elution system; and
   vii) a square carabiner that locks the sliding bolt latch in place;
wherein the floor frame cover holds a quick-release pin and a sliding bolt latch; and
wherein the said mobile adaptation kit securely holds the medical radioisotope elution system to the floor of a vehicle and ensures protection from any undesirable damage to the medical radioisotope elution system during transportation.

### BRIEF SUMMARY OF DRAWINGS

Further features and advantages of the present invention will become apparent from the following detailed description, taken in combination with the appended drawings, in which:

FIG. 1 shows a diagram of a schematically top view of a floor mounting system demonstrating principal elements in accordance with an embodiment of the present invention.

FIG. 2 shows a diagram of a schematically top diagonal view of the floor mounting system of FIG. 1 demonstrating principal elements in accordance with an embodiment of the present invention.

FIG. 3 shows a diagram of a schematically front view of the floor mounting system of FIG. 1 demonstrating principal elements in accordance with an embodiment of the present invention.

FIG. 4 shows a diagram of a schematically side view of the floor mounting system in accordance with another embodiment of the present invention.

FIG. 5A is a diagram of an elution system located over a floor mounting system in accordance with another embodiment of the present invention.

FIGS. 5B-H illustrate diagrams of various views of an elution system located over a floor mounting system, which is placed on a vehicle floor.

FIGS. 6A-6B illustrate the diagrams of T-bar attached to the side plates, which accommodate the mobile medical radioisotope elution system to transport safely and securely.

FIGS. 7A-7D illustrate the different components of the floor frame assembly (100) to accommodate the medical radioisotope elution or infusion system for support.

FIG. 7A depicts the floor frame serves as the foundational structure of the mobile adaptation kit, which can be permanently installed within the transport vehicle to provide a stable and secure base for the entire assembly.

FIG. 7B illustrates the isolated top view of the floor frame assembly.

FIG. 7C illustrates side view of the floor frame assembly.

FIG. 7D illustrates the bottom view of the floor frame assembly before fitting on the Van floor.

FIG. 8 illustrates the front (anterior) view of the floor frame assembly identifying the entry path of T-bar and the elution or infusion system.

FIG. 9 (9A and 9B) illustrates the side view of the damping system found inside the floor frame assembly.

FIG. 9A shows the pivoting piece at it's lowest point when the elution or infusion system is not docked inside.

FIG. 9B shows the pivoting piece in the raised position when the elution or infusion system is docked inside.

FIG. 10 illustrates the initial position or alignment of the floor frame assembly and the elution or infusion system before docking with each other.

FIGS. 11-16 show the fittings and docketing of the elution or infusion system inside the floor frame assembly from different positions.

### DETAILED DESCRIPTION

Exemplary embodiments are described in the following description with reference to the drawings. The drawings are not necessarily true to scale but are rather intended to schematically illustrate examples of the particular features. It should be noted that the features and components described in the following can be combined with each other independently of whether they were described in conjunction with a single embodiment. The combination of features in the respective embodiments serves only to illustrate the basic construction and the function of the claimed device.

The present disclosure provides a medical radioisotope transport system that securely holds the mobile medical radioisotope elution system inside a vehicle and ensures protection of the elution system during transportation and use.

The present disclosure provides a medical radioisotope transport system that securely holds the mobile medical radioisotope elution system to the floor of a vehicle and ensures protection of the elution system during transportation and use.

The medical radioisotope transport system or equipment as per the present invention is interchangeable with the term “mobile adaptation kit” or “mobile adaptation setup” or “mobile adaptation kit setup” or “mobile adaptation kit assembly”. The terms used herein such as trailers, van, truck, vehicle, are interchangeable and should be meant in a way to encompass any vehicle which is used or suitable for the transportation/delivery/movement/loading/unloading of medical elution system.

As used herein, the term “floor frame assembly” is synonymous to the term “FlexDock assembly.” The floor frame assembly generally comprises a floor frame configured to be attached to the transport vehicle floor permanently or temporarily; a floor frame cover with a top portion that creates the slot to install the T-bar into and out of the floor frame assembly; an angled rail that installs under the floor frame cover; a pivoting piece at the centre of the floor frame assembly; a primary lock system, which is a quick-release pin with a high-capacity pin that locks one end of the T-bar in place with the floor frame; and a secondary lock system, which is a sliding bolt latch along with square carabiner at another end of the floor frame to protect the T-Bar in position, in order to prevent sliding out of the radioisotope elution system.

As used herein, the “mobile medical radioisotope elution system” according to the present invention comprises one or more systems for dispensing a radioactive dose.

As used herein, the “mobile medical radioisotope elution and/or infusion transport system” according to the present invention can be synonymously referred hereinafter as the “medical vehicle system” or “vehicle” or “truck” or “trailer” or “van” or the like.

From the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

In an embodiment according to the present invention, there is provided a system comprising an integrated locking mechanism including combined bar, bolt, padlock and strap.

In an embodiment according to the present invention, there is provided a medical radioisotope transport system comprising:
a floor-mounted device in transport vehicle configured to support mobile medical radioisotope elution system;
a support mechanism with transportation plates configured to support the mobile medical radioisotope elution system; and
a locking device configured to securely and tightly connect the floor-mounted device and the support mechanism with transportation plates.

In an embodiment according to the present invention, there is provided a medical radioisotope transport system comprising:
a floor-mounted device in transport vehicle configured to support mobile medical radioisotope elution system;
a support mechanism with transportation plates configured to support the mobile medical radioisotope elution system; and
a locking device configured to securely and tightly connect the floor-mounted device and the support mechanism with transportation plates;
wherein the said transport system securely holds the medical radioisotope elution system to the floor of a vehicle and ensures protection from any undesirable damage to the medical radioisotope elution system during transportation.

In an embodiment according to the present invention, there is provided a medical radioisotope transport system comprising:
a floor-mounted device in transport vehicle configured to support mobile medical radioisotope elution system;
a support mechanism with transportation plates configured to support the mobile medical radioisotope elution system;
a locking device configured to securely and tightly connect the floor-mounted device and support mechanism with transportation plates;
the floor-mounting device is configured to further consist of a handle, at least two system plates, a floor frame, and at least a metal bar with a padlock to secure the equipment to the docking station; and
the mobile medical radioisotope elution system is configured to store at least one or more doses of nuclear medicine;
wherein the said transport system securely holds the mobile medical radioisotope elution system to the floor of a vehicle and ensures protection of the mobile medical radioisotope elution system with a radioisotope generator during transportation and wherein the said transport system is free of any tie-down straps or rings to hold the mobile medical radioisotope elution system within a vehicle.

In an embodiment according to the present invention, there is provided a strontium-rubidium radioisotope elution transport system comprising:
a floor-mounted device in a transport vehicle configured to support radioisotope elution system;
a support mechanism with transportation plates configured to support the radioisotope elution system;
a locking device configured to securely and tightly connects floor-mounted device and support mechanism with transportation plates;
the floor-mounting device is configured to further consist of a handle, at least two system plates, a floor frame, and at least a metal bar with a padlock to secure the equipment to the docking station; and
the radioisotope elution system is configured to store at least one or more doses of nuclear medicine;
wherein the said transport system securely holds the strontium-rubidium elution system to the floor of a vehicle and ensures protection of the elution system with a radioisotope generator during transportation and wherein the said transport system is free of any tie-down straps or rings to hold the radioisotope elution system within a vehicle.

In an embodiment according to the present invention, the floor-mounting device comprises at least one or more locking frames with a handle, system plate, floor frame, and one or more bolts.

In an embodiment according to the present invention, the floor-mounting device comprises at least one or more locking frames with a handle, system plate, floor frame, and a metal bar with a padlock to secure the equipment to the docking station.

In an embodiment according to the present invention, the mobile medical radioisotope elution system comprises Rb-82, O-15, F-18, Ga-68, Cu-61, C-11, N-13, Co-55, and Zr-89. In an embodiment according to the present invention, the mobile medical radioisotope elution system comprises Lu-177, Re-188, Ac-224, Zr-89, and Ra-223 isotope compounds. In a preferred embodiment according to the present invention, the mobile medical radioisotope elution system comprises a strontium-rubidium elution system.

In an embodiment according to the present invention, the radiopharmaceutical elution has source of radiopharmaceutical selected from Technetium-99m (^{99m}Tc), Iodine-123 (¹²³I), Iodine 124 (¹²⁴I), Iodine-125 (¹²⁵I), Iodine-131 (¹³¹I), Sodium Iodide (¹²³I, ¹²⁴I, ¹²⁵I, ¹³¹I), Phosphorous-32 (³²P), Indium-111 (¹¹¹ In), Cobalt-57 (⁵⁷Co), Erbium-169 (¹⁶⁹Er), Gallium-67 (⁶⁷Ga), Gallium-68 (⁶⁸Ga), Ammonia-13 (¹³N), Sodium fluoride (¹⁸F), Flourine-18 (¹⁸F), Lutetium Lu-177 (¹⁷⁷Lu), Radium-223 (²²³Ra), Samarium-153 (¹⁵³Sm), Thallium-201 (²⁰¹Tl), Yttrium-90 (⁹⁰Y), Chromium-51 (⁵¹Cr), Holmium-166 (¹⁶⁶Ho), Rhenium-186 (¹⁸⁶Re), Rhenium-188 (¹⁸⁸Re), Rubidium-81 (⁸¹Rb), Strontium-89 (⁸⁹Sr), Strontium-82 (⁸²Sr), Actinium-225 (²²⁵Ac), Xenon-133 (Xe-133), and Gold-198 (¹⁹⁸Au).

In an embodiment according to the present invention, the radiopharmaceutical dispensing system may include various components like a controller, pumps, valves, sensors, radiopharmaceutical source, cabinet, shielded assembly, shielded chambers, tubing, activity detector, dose calibrator, computer, internal memory, user interface, motors, syringes, pipettes, needles, exhaust, filters, and other accessories. Radiopharmaceutical dispensing systems may be connected to one or more equipment or networks like imaging systems, infusion systems, hospital networks, pharmacies, servers, remote computers, iPads, mobile tablets, mobile phones, watches, or like. The radiopharmaceutical dispensing system may be connected via wired or wireless connections like Local Area Network (LAN), internet, communication ports, Wireless Fidelity (Wi-Fi), Universal Serial Bus (USB), Bluetooth, Cables, Compact Disc, Digital Video Disc and/or combinations thereof.

In an embodiment according to the present invention, the system permits one to transport mobile medical radioisotope elution system with a radioisotope generator in a vehicle such as a truck or a van, or a trailer.

In an embodiment according to the present invention, the transport system is free of any tie-down straps used to hold the mobile medical radioisotope elution system within a vehicle.

In an embodiment according to the present invention, the transport system is free of any mechanism, assembly or setup wherein the mobile medical radioisotope elution system is fixed using straps to a bracket located on the floor of the vehicle.

In an embodiment according to the present invention, the transport system is free of any mechanism, assembly or setup wherein the mobile medical radioisotope elution system is fixed using straps, elastic, rope, belt, hook, buckle or ratchet to a bracket located on the floor of the vehicle.

In an embodiment according to the present invention, the transport system is free of any tie-down rings used to hold the mobile medical radioisotope elution system with a radioisotope generator within a vehicle.

In an embodiment according to the present invention, the transport system is free of any tie-down wires used to hold the mobile medical radioisotope elution system with a radioisotope generator within a vehicle.

In an embodiment according to the present invention, the transport system is used to permanently lock the mobile medical radioisotope elution system in a vehicle such as a truck, van, or trailer.

In an embodiment according to the present invention, the transport system enables the mobile medical radioisotope elution system for use in both fixed trailers, van or truck and mobile on-site delivery as per hospital or patient's need.

In an embodiment according to the present invention, the transport system is used to load or unload the mobile medical radioisotope elution system from a vehicle such as a truck, van, or trailer based on the need of the user.

In an embodiment according to the present invention, the transport system is used to load the mobile medical radioisotope elution system by fixing it on a frame and securing it with a locking-unlocking device.

In an embodiment according to the present invention, the transport system comprises a plurality of wheels configured with brake mechanisms. In another embodiment according to the present invention, the transport system comprises a plurality of wheels configured without brake mechanisms.

In an embodiment according to the present invention, the transport system comprises a shock-absorbing mechanism.

In an embodiment according to the present invention, the mobile medical radioisotope elution system further comprises a locking device to secure the radioisotope inside the elution system.

In an embodiment according to the present invention, the mobile medical radioisotope elution system further comprises a locking device for the generator lead well.

In an embodiment according to the present invention, the mobile medical radioisotope elution system comprises a device for securing the dose calibrator during transportation.

In an embodiment according to the present invention, the mobile medical radioisotope elution system comprises an arrangement for securing the computer, all hardware, and all software of the elution system during transportation.

In an embodiment of the present invention, the mobile adaptation kit is installed in and carried by a transport system or a medical vehicle, wherein the transport system or medical vehicle is selected from the group consisting of medical truck, medical van, medical trailer, bus, or any kind of medical vehicle suitable to carry medical equipments along with patients and other crew members.

In an embodiment of the present invention, the medical radioisotope elution system or Radioisotope generator system or vehicle comprises a plurality of sensor systems. The sensor system is selected from the group consisting of a temperature sensor, pressure sensor, motion sensor, vibration sensor, current sensor, speed sensor, air detection sensor, occlusion detection sensor, humidity sensor, position sensor, a wind sensor, a wave sensor, an engine control sensor, an electromagnetic field sensor, and voltage sensor. The said sensors may be connected to an electronics system of the vehicle and, in particular, the vehicle may be configured to be operated based on sensor readings obtained from the various sensors. In an embodiment of the present invention, the sensor system of the vehicle is configured to be in communication with the controller, which is further configured to be in communication with a computer system.

In an embodiment of the present invention, the mobile adaptation kit further comprises a plurality of sensor systems. The sensor system is selected from the group consisting of a temperature sensor, pressure sensor, motion sensor, vibration sensor, current sensor, speed sensor, air detection sensor, occlusion detection sensor, humidity sensor, position sensor, a wind sensor, a wave sensor, and voltage sensor. The sensor system also can include a location sensor so that the vehicle can be tracked from a different location, such as a central office from which one or more of the vehicles are based.

In an embodiment according to the present invention, the transport system further comprises an alarm system. In another embodiment, the alarm system shall provide audible and/or visual alerts/alarms to the operator or user. In case any problem or undesirable activity in the elution system occurs such as any leakage, damage, missing of any part, any default in any part of the device, the system will provide the alert and/or alarm to the operator.

In an embodiment, the alarm system shall provide continuously audible and/or visual alerts/alarms to the operator or user in case the elution system is not properly or loosely connected with the transport system.

In an embodiment according to the present invention, the mobile medical radioisotope elution or infusion transport system comprises a navigation and/or tracking systems such as GPS (global positioning system) device which provide accurate position, velocity, and time information of vehicles or elution system. In another embodiment, the tracking system simultaneously can provide real-time data such as accurate position, velocity, and time information of vehicles or elution system to multiple locations such as loading location, delivery location, radiopharmacy, or hospital location. In an embodiment, the navigation and/or tracking system of mobile medical radioisotope elution system can be connected to the GPS system of a truck or van, or trailer.

In an embodiment of the present invention, the mobile medical radioisotope elution/infusion transport system or medical vehicle system comprises one or more of an inertial measurement unit (IMU), a vehicle navigation system, an autonomous driving system, a board computer, a compass, a radar, and a lidar.

In an embodiment of the present invention, there is provided a mobile medical radioisotope elution and/or infusion transport system, hereinafter referred to as the “medical vehicle system”. The medical vehicle system is configured to facilitate the safe and efficient transport and administration of medical radioisotopes for diagnostic imaging in remote or underserved locations.

In an embodiment of the present invention, the medical vehicle system comprises:
a) an inertial measurement unit (IMU) for detecting and monitoring vehicle orientation and motion; and accurately and objectively monitoring of patient movement;
b) a vehicle navigation system for route planning and geolocation;
c) a fully or partially autonomous driving system configured to enable self-directed navigation and obstacle avoidance;
d) an onboard computing unit for system control, data processing, and integration of subsystems;
e) a compass for directional orientation;
f) a radar system for object detection and environmental mapping; and
g) a lidar system for high-resolution spatial awareness and navigation support.

In an embodiment of the present invention, the mobile medical radioisotope transport system comprises:
a power supply system comprising an on-board generator and battery backup configured to supply continuous power to medical imaging and diagnostic equipment, thereby enabling operation independent of external power infrastructure or connection to an external power infrastructure;
a climate control system configured to regulate internal environmental parameters to ensure optimal operational conditions for sensitive imaging equipment and to maintain patient comfort during diagnostic or therapeutic procedures;
a data transmission system configured to enable secure and efficient transmission of imaging and diagnostic data to remote diagnostic centers or healthcare facilities, thereby facilitating real-time consultation and analysis; and
a patient interface system comprising an accessible entry mechanism for patient ingress and egress, interior accommodations designed for patient positioning and comfort, and a digital interface configured to facilitate patient registration, consent acquisition, and interaction with healthcare personnel.

In an embodiment of the present invention, the climate control system is configured to maintain a temperature within the transport or vehicle system between 18° C. and 24° C. and relative humidity below 60%.

In an embodiment of the present invention, the mobile medical radioisotope transport system or vehicle system further comprises a Picture Archiving and Communication System (PACS) integrated with cloud-based storage to store, retrieve, manage, and share medical images.

In an embodiment of the present invention, the patient interface system includes a wheelchair lift and adjustable seating for patient comfort.

In an embodiment of the present invention, the mobile medical radioisotope transport system or vehicle system further comprises an artificial intelligence module configured to assist in preliminary image analysis.

In an embodiment of the present invention, the mobile medical radioisotope transport system or vehicle system further is configured for deployment in rural or disaster-affected areas. In such applications, the use of a location tracking sensor may be helpful to provide information to the vehicle to avoid impassable locations in disaster-affected areas.

In an embodiment of the present invention, the mobile medical radioisotope transport system or vehicle system comprises a method for providing mobile medical imaging services, comprising:
transporting a vehicle equipped with at least one medical imaging modality to a predetermined or designated location for diagnostic service delivery;
stabilizing the vehicle at the location using an integrated sensor system configured to assess terrain and adjust vehicle positioning for optimal operational stability;
activating an onboard power supply subsystem, comprising a generator and battery backup, to initiate and sustain operation of the medical imaging modality selected from the group consisting of PET, PET/CT, dynamic PET, SPECT, SPECT/CT, MRI, CT, ultrasound-CT, PET/MRI and the like;
initiating the vehicle sensor system to monitor environmental and operational parameters during imaging procedures;
registering a patient through a digital interface configured to capture patient identification, medical history, and consent documentation;
performing a diagnostic imaging procedure using the imaging modality selected from the group consisting of PET, PET/CT, dynamic PET, SPECT, SPECT/CT, MRI, CT, ultrasound-CT, PET/MRI and the like;
transmitting the acquired imaging data to a remote diagnostic center via a secure data transmission subsystem for expert analysis and interpretation; and
receiving and storing diagnostic results within the onboard data management system for subsequent clinical use, patient consultation, or integration into electronic health records.

In one embodiment of the present invention, the medical vehicle comprises one or more enclosed or semi-enclosed sections, cubicles, or rooms, each configured to house a medical imaging system. The medical imaging system may be selected from a group consisting of positron emission tomography (PET), PET/computed tomography (PET/CT), dynamic PET, single-photon emission computed tomography (SPECT), SPECT/CT, magnetic resonance imaging (MRI), computed tomography (CT), ultrasound-CT, PET/magnetic resonance imaging (PET/MRI) and other equivalent or analogous imaging modalities.

The medical vehicle further comprises a mobile adaptation kit, structurally integrated with the vehicle via a floor frame assembly. The floor frame assembly is configured to securely support an elution and/or infusion system, which includes a radioisotope generator, a fluid pump, a dose calibrator, a waste containment unit, and other essential components housed within shielded compartments to ensure radiation safety and operational integrity.

Each cubicle referenced above may be dimensioned to accommodate the respective medical imaging system, with optional additional space for operator access, patient positioning, or auxiliary equipment. The cubicles may be defined by physical partitions or alternatively by virtual boundaries within the volumetric space of the vehicle's interior.

Furthermore, the medical vehicle is designed with sufficient internal capacity to accommodate a minimum of 15 individuals simultaneously, exclusive of equipment and storage provisions. In a preferred embodiment, the vehicle may accommodate between 15 and 20 persons concurrently, thereby enabling efficient workflow, patient throughput, and staff coordination during mobile diagnostic operations. It should be understood that it is not necessary that 15 individuals be present simultaneously as the number of individuals present may vary depending upon the particular circumstances.

In an embodiment of the present invention, the mobile adaptation kit may accommodate any of the mobile medical device known in the state of the art. The mobile medical device can be selected from the group of any mobile medical radioisotope generator system, radioisotope elution system and/or radioisotope infusion system, wherein the devices are selected from the group consisting of ⁸²Sr/⁸²Rb, ¹⁰Cd/^{109m}Ag, ¹¹⁸Te/¹¹⁸Sb, ¹³⁷Cs/^{137m}Ba, ¹³⁴Ce/¹³⁴La, ¹⁴⁴Ce/¹⁴⁴Pr, ¹⁴⁰Nd/¹⁴⁰Pr, ¹⁶⁷Tm/^{167m}Er, ¹⁷⁸W/¹⁷⁸Ta, ¹⁹¹Os/^{191m}Ir, ⁹⁹Mo/^{99m}Tc, ⁶⁸Ge/⁶⁸Ga, and variations thereof. Further, the mobile adaptation kit may accommodate any of the mobile medical devices include PET, PET/CT, dynamic PET, SPECT, SPECT/CT, MRI, CT, ultrasound-CT, PET/MRI and the like. The mobile adaptation kit may accommodate F-18 flyrcado for positron emission tomography (PET) myocardial perfusion imaging in adults to detect coronary artery disease (CAD).

In an embodiment, the navigation and/or tracking system of the mobile medical radioisotope elution system can provide audible and/or visual alerts/alarms to the operator or user, in case there is any change and/or error in route direction.

In an embodiment, the navigation and/or tracking system enable the users to share audible and/or visual alerts/alarms or messages to each connected user located at various locations. In an embodiment, the system is equipped with a vehicle navigation system selected from the group consisting of one or more of microprocessor modules, LCD touch control display module, Access Port module, GPRS wireless communication module, GPS global positioning module, data memory module, Bluetooth module, LCD touch control display module, and/or wireless sensor network module electrically connected.

In an embodiment according to the present invention, the mobile medical radioisotope transport or elution system comprises a damage avoidance system that is activated once the risk of damage is detected, before impact with the surface or air. To illustrate a specific example, the mobile medical radioisotope elution system is equipped with a damage avoidance system that includes a safety monitoring system and/or a protection system. In this sense, a damage avoidance system comprises one or more protection elements working together to reduce or prevent damage to the elution system on hitting the surface or any hard object. In an embodiment, the protection system deploys an airbag before the elution system hits any hard object or surface. The airbag will absorb the impact and protect the system to reduce or eliminate damage.

In an embodiment according to the present invention, the medical radioisotope transport system maintains the system functionality as per the manufacturer's specification throughout the loading-unloading and transportation process.

In an embodiment according to the present invention, the medical radioisotope transport system secures the generator, computer, waste bottle, and other critical components throughout the loading-unloading and transportation process.

In an embodiment according to the present invention, the transport system further comprises a locking-unlocking system.

In another embodiment, the locking-unlocking system is a lock and key-based system.

In another embodiment, the locking-unlocking system is a numbering-based system.

In another embodiment, the locking-unlocking system is a biometric-based system.

In another embodiment, the biometric locking-unlocking system is operated through fingerprints, thumbprints, retina of operator or user, or like.

In an embodiment according to the present invention, the transport system further comprises a power backup system configured to provide power with at least one power storage device.

In an embodiment according to the present invention, the transport system further comprises a monitoring device navigation system to estimate the location of the system.

In an embodiment according to the present invention, the transport system further comprises a monitoring device configured to connect to a wireless network and exchange information across the wireless network to the mobile medical radioisotope elution system.

In an embodiment according to the present invention, the system plates comprise at least two plates located on the left and right sides of the floor-mounting device.

In an embodiment according to the present invention, the one or more bolts used in the floor-mounting device are U-bolts.

In another embodiment, the system comprises a metal bar with a padlock to secure the equipment to the docking station.

In an embodiment according to the present invention, there is provided a strontium-rubidium radioisotope elution transport system comprising one or more adaptation kits, a frame locking system, generator door locking system, generator padding, computer stabilization, dose calibrator locking, and waste bottle.

In an embodiment according to the present invention, there is provided a strontium-rubidium elution system comprising an attachment for engaging the elution system over the mounting system, wherein the mounting system is attached or fixed to the base of the vehicle.

In an embodiment according to the present invention, there is provided a method to transport a strontium-rubidium elution system with enhanced safety features, wherein the method comprises complete quality control testing of the elution system after completion of transport with one or more tests, including but not limited to, visual inspection, and installation qualification test.

In another embodiment, the technical integrity of the elution system is tested using one or more following methods: a) verifying all tubings remain properly installed with the strontium-rubidium elution system; b) verifying pressure transducer remains plugged into the strontium-rubidium elution system; c) verifying that all valves e.g. pinch valves are intact or not damaged; d) verifying that tubing within the activity counter is properly installed and the door is properly closed; e) verifying that pump is intact or not damaged, and f) verifying that UPS is powered on.

In an embodiment according to the present invention, a mobile adaptation kit may comprise:
a) a floor frame assembly installed in a transport vehicle configured to support a mobile medical radioisotope elution system; and
b) a T-bar that slides inside and out of the floor frame assembly, wherein the T-bar comprises a gasket and nylon sheets;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail positioned on each side of the floor frame assembly that installs under the floor frame cover;
   iv) a pivoting piece at the centre of the floor frame assembly;
   v) a quick-release pin with a high-capacity pin that locks one end of the T-bar in place with the floor frame; and
   vi) a locking mechanism that locks at one end of the T-Bar in position, in order to prevent sliding out of the radioisotope elution system;
wherein the gasket helps to absorb vibration during transportation; and the nylon sheets help to reduce friction when inserting into the floor frame assembly.

In an embodiment according to the present invention, the mobile adaptation kit comprises:
a) a floor frame assembly installed in a transport vehicle configured to support a mobile medical radioisotope elution system; and
b) a T-bar that slides inside and out of the floor frame assembly, wherein the T-bar comprises a gasket and nylon sheets;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail positioned on each side of the floor frame assembly that installs under the floor frame cover;
   iv) a pivoting piece at the centre of the floor frame assembly, installed with high load compression springs on one end;
   v) a quick-release pin with a high-capacity pin that locks one end of the T-bar in place with the floor frame; and
   vi) a locking mechanism that locks at one end of the T-Bar in position, in order to prevent sliding out of the radioisotope elution system;
wherein the gasket helps to absorb vibration during transportation; and the nylon sheets help to reduce friction when inserting into the floor frame assembly; and
wherein the said mobile adaptation kit securely holds the medical radioisotope elution system to the floor of a vehicle and ensures protection from any undesirable damage to the medical radioisotope elution system during transportation.

In an embodiment according to the present invention, the mobile adaptation kit comprises:
a) a floor frame assembly installed in a transport vehicle configured to support a mobile medical radioisotope elution system; and
b) a T-bar that slides inside and out of the floor frame assembly, wherein the T-bar comprises a gasket and nylon sheets; and wherein the gasket helps to absorb vibration during transportation; and the nylon sheets help to reduce friction when inserting into the floor frame assembly;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail positioned on each side of the floor frame assembly that installs under the floor frame cover, wherein the angled rail is used as a guide to align the T-bar into position;
   iv) a pivoting piece at the centre of the floor frame assembly, installed with high load compression springs on one end, wherein the pivoting piece gets activated when the T-bar is in the final position;
   v) a quick-release pin with a high-capacity pin that locks one end of the T-bar in place with the floor frame;
   vi) a sliding bolt latch that locks other end of the T-Bar in position, in order to prevent sliding out of the radioisotope elution system; and
   vii) a square carabiner that locks the sliding bolt latch in place;
wherein the floor frame cover holds a quick-release pin and a sliding bolt latch; and
wherein the said mobile adaptation kit securely holds the medical radioisotope elution system to the floor of a vehicle and ensures protection from any undesirable damage to the medical radioisotope elution system during transportation.

In an embodiment according to the present invention, the mobile adaptation kit comprises:
a) a floor frame assembly installed in a transport vehicle configured to support a mobile medical radioisotope elution system; and
b) a T-bar that slides inside and out of the floor frame assembly, wherein the T-bar comprises a gasket and nylon sheets;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail positioned on each side of the floor frame assembly that installs under the floor frame cover;
   iv) a pivoting piece at the centre of the floor frame assembly, installed with high load compression springs on one end;
   v) a quick-release pin with a high-capacity pin that locks one end of the T-bar in place with the floor frame; and
   vi) a locking mechanism that locks at one end of the T-Bar in position, in order to prevent sliding out of the radioisotope elution system;
wherein the gasket helps to absorb vibration during transportation; and the nylon sheets help to reduce friction when inserting into the floor frame assembly;
wherein the said mobile adaptation kit securely holds the medical radioisotope elution system to the floor of the transport vehicle and ensures protection from any undesirable damage to the medical radioisotope elution system during transportation;
wherein the transport vehicle further comprises:
a) at least one cubicle within the vehicle, the cubicle being dimensioned to accommodate a medical imaging;
b) an inertial measurement unit (IMU) for detecting and monitoring vehicle orientation and motion; and accurately and objectively monitoring of patient movement;
c) a power supply system comprising an on-board generator and battery backup configured to supply continuous power to medical imaging and diagnostic equipment; and
d) a sensor system; wherein the sensor system includes one or more sensors selected from the group consisting of a temperature sensor, pressure sensor, motion sensor, vibration sensor, current sensor, speed sensor, air detection sensor, occlusion detection sensor, humidity sensor, position sensor, a wind sensor, a wave sensor, an engine control sensor, an electromagnetic field sensor, and voltage sensor or combination thereof.

In an embodiment according to the present invention, the mobile adaptation kit comprises:
a) a floor frame assembly installed in a transport vehicle configured to support a mobile medical radioisotope elution system; and
b) a T-bar that slides inside and out of the floor frame assembly, wherein the T-bar comprises a gasket and nylon sheets; and wherein the gasket helps to absorb vibration during transportation; and the nylon sheets help to reduce friction when inserting into the floor frame assembly;
wherein the floor frame assembly comprises:
   i) a floor frame that is installed and remains within the vehicle or van;
   ii) a floor frame cover with a top portion that creates the slot to install the T-bar inside and out of the floor frame assembly;
   iii) an angled rail on each side of the floor frame assembly that installs under the floor frame cover, wherein the angled rail is used as a guide to align the T-bar into position;
   iv) a pivoting piece at the centre of the floor frame assembly, installed with high load compression springs on one end, wherein the pivoting piece is activated when the T-bar is in the final position;
   v) a quick-release pin with a high-capacity pin that locks one end of the T-bar in place with the floor frame;
   vi) a sliding bolt latch that locks the other end of the T-Bar in position to prevent sliding out of the radioisotope elution system; and
   vii) a square carabiner that locks the sliding bolt latch in place;
wherein the floor frame cover holds a quick-release pin and a sliding bolt latch;
wherein the said mobile adaptation kit securely holds the medical radioisotope elution system to the floor of a vehicle and ensures protection from undesirable damage to the medical radioisotope elution system during transportation; and
wherein the mobile adaptation kit further comprises a sensor system; wherein the sensor system is selected from a group consisting of one or more sensors such as a temperature sensor, pressure sensor, motion sensor, vibration sensor, self-levelling sensor, current sensor, speed sensor, power supply sensor, air detection sensor, occlusion detection sensor, humidity sensor, position sensor, a wind sensor, a wave sensor, and voltage sensor or combination thereof.

In an embodiment according to the present invention the mobile adaptation kit further comprises one or more torx screws, and one or more ball bearings for shaft.

In an embodiment according to the present invention, the mobile medical radioisotope elution system comprises a strontium-rubidium elution system.

In an embodiment according to the present invention, the pivoting piece further comprises a high load fastener mount compression spring.

In an embodiment according to the present invention, the high load fastener mount compression spring is sized to hold a weight from about 100 kg to about 800 kg.

In another embodiment according to the present invention, the pivoting piece comprises pivoting shaft and flange locknut.

In an embodiment according to the present invention, the locking mechanism comprises a sliding bolt latch; and a square carabiner that locks the sliding bolt latch in place; wherein the sliding bolt latch is used to prevent sliding out of the radioisotope elution system.

In an embodiment according to the present invention, the angled rail is used as a guide to align the T-bar into position.

In an embodiment according to the present invention, the pivoting piece is activated when the T-bar is in the final position.

In an embodiment according to the present invention, the high load fastener mount compression springs are used to dampen the vibration during transit and minimize the vibration impact on a radioisotope elution system.

In an embodiment according to the present invention, the adaptation kit further comprises side plates that attach to the radioisotope elution system.

In an embodiment according to the present invention, the adaptation kit further comprises one or more socket head screws.

In an embodiment according to the present invention, the pivoting piece further comprises one or more external retaining rings.

In an embodiment according to the present invention, the mobile adaptation kit further comprises one or more screws, and one or more washers.

In an embodiment according to the present invention, the locking mechanism further comprises a latch strike-plate; a latch housing; a latch assembly; a nylon-insert locknut; and a flat head torx screw.

In an embodiment according to the present invention, the latch assembly comprises a latch body; a latch handle; and a socket head screw.

In an embodiment according to the present invention, the sliding bolt latch locks other end of the T-bar in position to prevent sliding out of the radioisotope elution system.

In an embodiment according to the present invention, the mobile adaptation kit secures the generator, computer, waste bottle, and other critical components throughout the transportation process.

In an embodiment according to the present invention, the mobile adaptation kit further comprises a sensor system; wherein the sensor system comprises at least one or more sensors selected from a group consisting of a temperature sensor, pressure sensor, motion sensor, vibration sensor, current sensor, speed sensor, air detection sensor, occlusion detection sensor, humidity sensor, position sensor, voltage sensor or combination thereof.

In an embodiment according to the present invention, the mobile adaptation kit is attached at the floor of a mobile medical radioisotope transport system or medical vehicle, wherein the mobile medical radioisotope transport system or medical vehicle comprises:
a vehicle structure configured to house one or more medical imaging systems; and
at least one cubicle within the vehicle, dimensioned to accommodate a medical imaging modality;
wherein the medical imaging system is selected from the group consisting of PET, PET/CT, dynamic PET, SPECT, SPECT/CT, MRI, CT, ultrasound-CT, PET/MRI and thereof;
wherein the cubicle is defined by physical walls or by a virtual boundary within the internal volume of the vehicle; and
wherein the cubicle includes additional space adjacent to the medical imaging system to facilitate operator access or patient positioning.

In an embodiment of the present invention, the mobile medical radioisotope transport system or transport vehicle comprises a method for providing mobile medical imaging services, the method comprising:
transporting a vehicle equipped with at least one medical imaging modality to a predetermined or designated location for diagnostic service delivery;
stabilizing the vehicle at the location using an integrated sensor system configured to assess terrain and adjust vehicle positioning for optimal operational stability;
activating an onboard power supply subsystem, comprising a generator and battery backup, to initiate and sustain operation of the medical imaging modality selected from the group consisting of PET, PET/CT, dynamic PET, SPECT, SPECT/CT, MRI, CT, ultrasound-CT, PET/MRI and the like;
initiating the vehicle sensor system to monitor environmental and operational parameters during imaging procedures;
registering a patient through a digital interface configured to capture patient identification, medical history, and consent documentation;
performing a diagnostic imaging procedure using the imaging modality selected from the group consisting of PET, PET/CT, dynamic PET, SPECT, SPECT/CT, MRI, CT, ultrasound-CT, PET/MRI and the like;
transmitting the acquired imaging data to a remote diagnostic center via a secure data transmission subsystem for expert analysis and interpretation; and
receiving and storing diagnostic results within the onboard data management system for subsequent clinical use, patient consultation, or integration into electronic health records.

In an embodiment of the present invention, the mobile adaptation kit ensures the quality under the United States Food and Drug Administrations (US FDA) guidelines and the Code of Federal Regulations (CFR) guidelines. The mobile adaptation kit of the present invention ensures the quality control tests, which includes usability testing, hardware testing, mobile trailers safety features, power supplies, power back-up, EMC and electrical safety testing, clinical simulation performance testing, breakthrough testing of the elution system, summative testing impact analysis, and/or essential performance impact assessment. Further, all information stated on each label must also be contained in each batch production record. The procedures must include review by a designated person to meet any of its specifications and an investigation to determine the quality of the product. The mobile adaptation kit of the present invention ensures all the quality control tests in accordance with applicable Federal and State law. Further, the onboard dose calibrator of the elution system or infusion system is remain in the same position when secured with the mobile adaptation kit during transportation in mobile PET imaging facility. The dose calibrator operates in a range of 2.59×103 Bq to 3.4706×105 MBq (0.07 μCi to 9.38 Ci) of ⁸²Rb with an accuracy of 10% in a mobile PET imaging facility. Moreover, in an embodiment of the present invention, the method to secure the elution or infusion system to the floor frame is capable of withstanding vibration profile in the Y-axis in accordance with ISTA (International Safe Transit Association) 3H profile (Random Vibration for air-ride trailers) and up to 4.6 gRMS shock in the X-axis (horizontal). The method of securing the floor to the mobile PET imaging facility is capable of withstanding vibration profile in the Y-axis in accordance with ISTA (International Safe Transit Association) 3H profile (Random Vibration for air-ride trailers) and up to 4.6 gRMS shock in the X-axis (horizontal). At 6% Duty Cycle, the elution or infusion system has an expected life time of 5 years in a mobile PET imaging facility environment (shall withstand vibrations up to 1.2 gRMS in the Y axis (Random Vertical) and shock up to 4.6 gRMS in X axis (Horizontal)) when serviced according to its preventive maintenance program. Other components like valves, pump system, monitor or user interface, computer are in proper position.

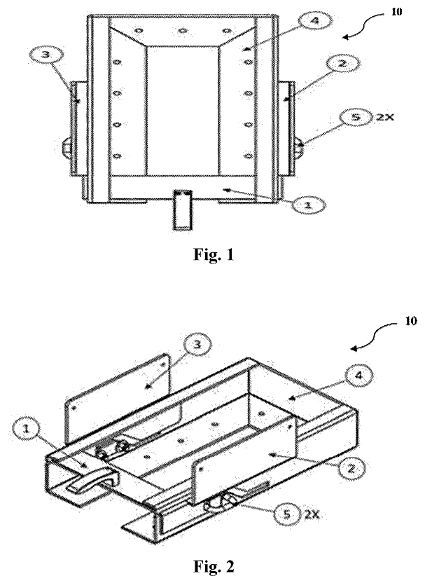

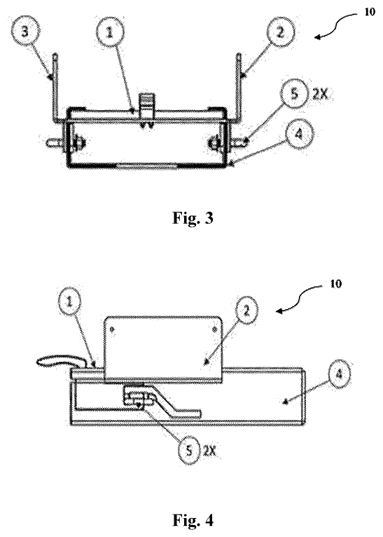

FIGS. 1-4 illustrate diagrams of floor mounting system (10), demonstrating principal elements in accordance with an embodiment of the present invention. The floor mounting system (10) includes a locking frame with a handle (1) that helps to move the elution system in a floor-mounted position. The floor mounting system (10) also includes a system plate left (2) and a system plate right (3) that help to hold and/or securely fix the elution system on the floor mounting system (10). A floor frame or docking station (4) holds the whole floor mounting system (10) to the floor of a vehicle/truck/van/trailer, etc. and a metal bar with a padlock (5) secures the elution system to the floor frame or docking station.

FIG. 4 illustrates diagrams of the side view of the floor mounting system (10) and diagonal view of a locking plate subassembly respectively which depict the mechanism of a metal bar with a padlock (5) to secure the elution system to the floor frame or docking station (4) effectively.

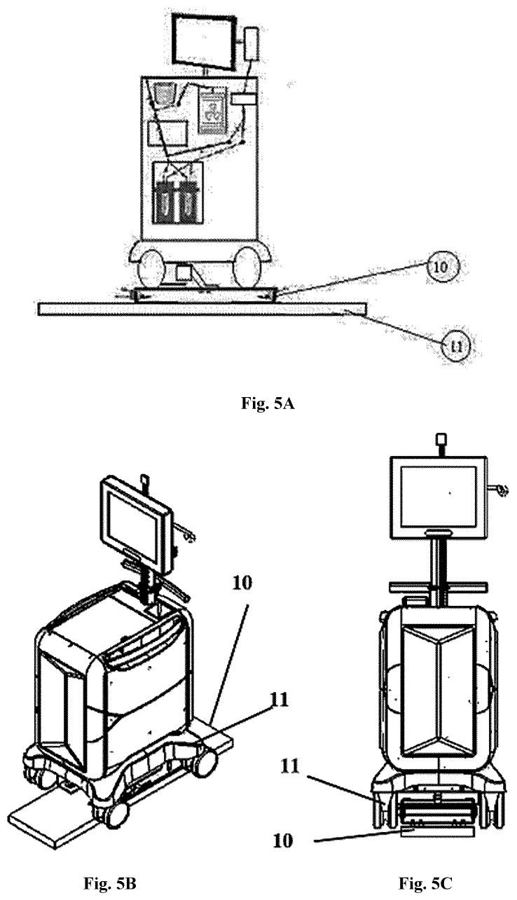

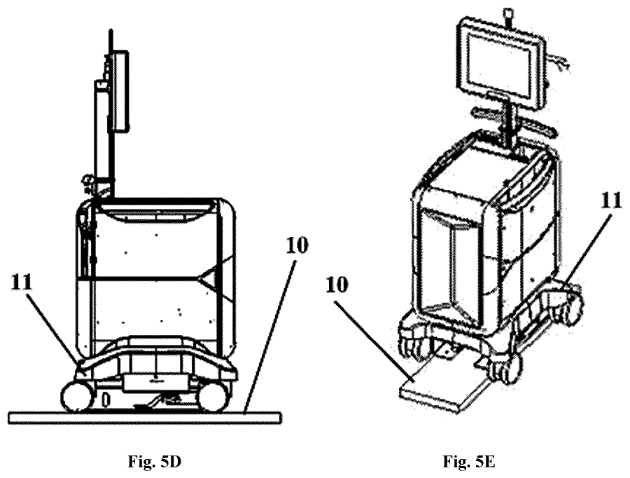

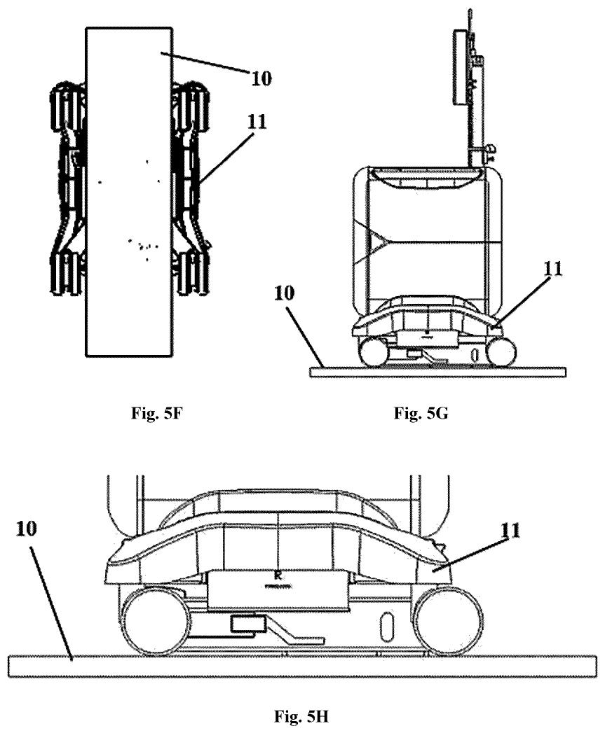

FIGS. 5A-H illustrate diagrams of various views of an elution system located over a floor mounting system (10) which is placed on a vehicle floor (11). The elution system slides over the mounting system, which is located or fixed on the floor of the vehicle and an engaging attachment is installed on the base of the elution system to get it locked with the mounting system via the lock handle.

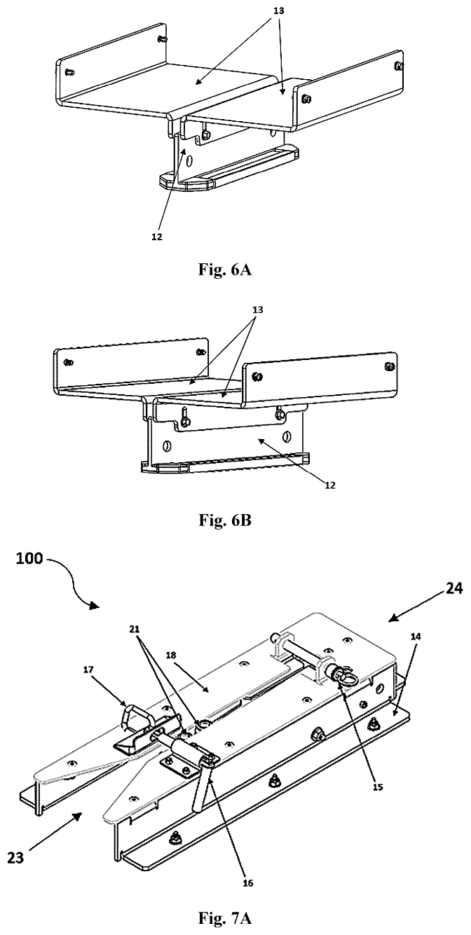

FIGS. 6A-6B illustrate the diagrams of T-bar attached to the side plates, which accommodate the mobile medical radioisotope elution system to transport safely and securely. The T-bar (12) has nylon or rubber like sheets to absorb the shocks or vibration during the transport. The T-bar (12) further is attached with the side plates (13), which accommodates the medical radioisotope elution system or any infusion system or radioisotope generator system.

Although the T-bar may have nylon or rubber-like sheets, other materials are suitable. For example, the T-bar may be aluminum but to address the interaction between two aluminum surfaces, the surfaces may be coated or configured in the form of a multilayer composite with outer, low friction, polymer surfaces intended to reduce the friction when siding the T-bar into its receiving slot. Aluminum or metal surfaces in the T-bar may have a nylon sheet applied, affixed or as part of a multilayer composite so that friction is reduced and a shock absorbing surface is created. Although nylon is a suitable polymer for such a surface, other polymers are suitable, including polypropylene, nylon, polyethylene, polytetrafluoroethylene (PTFE), polyethylene terephthalate (PET), poly-ether-ether-ketone (PEEK), and Acetal. Therefore where nylon is referenced, any other suitable, low friction polymer may be used in its place.

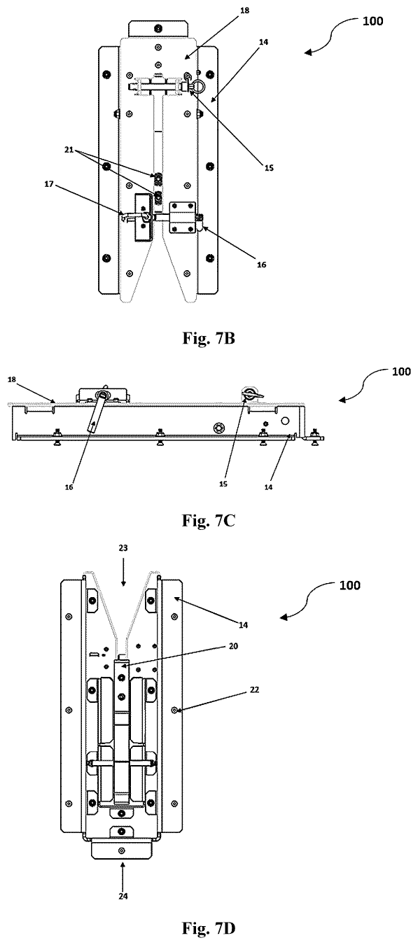

The side plates (13) one on each side, are designed to remain permanently attached to the mobile medical radioisotope elution system or any infusion system or radioisotope generator system, which ensures the stability and eliminating the need for removal during operation. These side plates (13) contribute to the system's structural integrity and simplify usage. The T-bar (12), a key sliding component within the floor frame (14) (as shown in the FIG. 7A) slides inside and out of the floor frame assembly (100). More particularly, the T-bar facilitates vertical displacement to accommodate custom flooring in transport vehicle. It comprises compression springs (21) to absorb vibrations and nylon sheets that minimize friction, allowing smooth insertion and removal while enhancing durability and performance.

FIGS. 7A-7D illustrate the different components of the floor frame assembly (100) to accommodate the medical radioisotope elution or infusion system for support. FIG. 7A depicts the floor frame (14) which serves as the foundational structure of the mobile adaptation kit, permanently installed within the transport vehicle to provide a stable and secure base for the entire assembly. Mounted atop this frame is the floor frame cover (18), which forms the upper surface and includes a precisely engineered slot for inserting and removing the T-Bar (12). This cover also houses the quick-release pin (15) and the sliding bolt latch (16), both of which are essential for securing the T-Bar (12) in place. FIG. 7A depicts the entry (23) for the T-bar (12) and the closed end portion (24) of the floor frame assembly (100). Under the floor frame cover (18) lies the angled rail (19) (as shown in FIGS. 9A and 9B) on each side, a guiding component that ensures accurate alignment of the T-bar (12) during installation.

FIG. 7B illustrates the isolated top view of the floor frame assembly (100), wherein the compression springs (21) are visible at the middle position under the floor frame cover (18). FIG. 7C illustrates side view of the floor frame assembly (100). FIG. 7D illustrates the bottom view of the floor frame assembly (100) before fitting on the Van or vehicle floor, wherein the entry path (23) and the closed portion (24) of the medical radioisotope elution or infusion system are clearly visible.

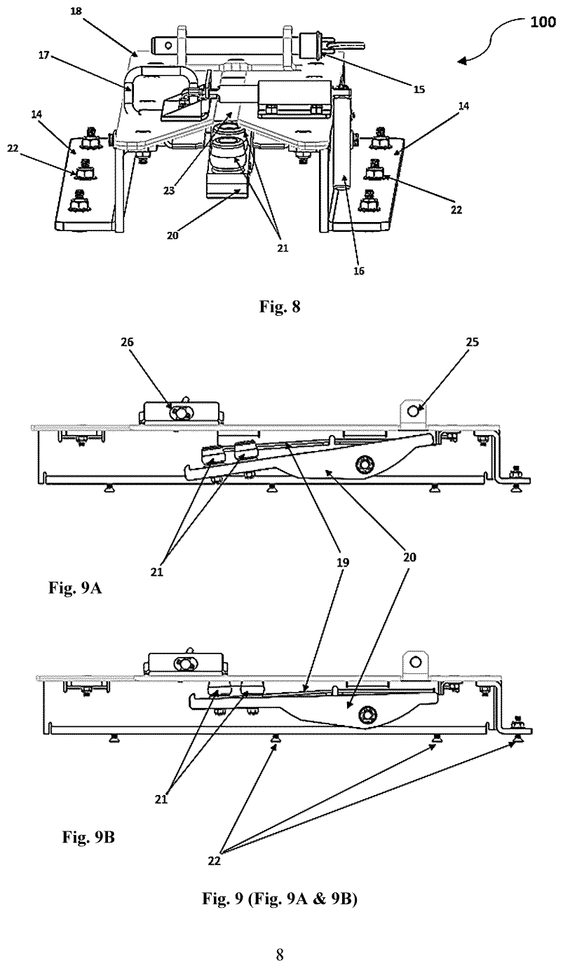

FIG. 8 illustrates the front (anterior) view of the floor frame assembly identifying the entry path (23) of T-bar and the elution or infusion system. Further, FIG. 8 depicts the one or more bolts and screws (22) used to fit the floor frame (14) and other parts and components of the entire floor frame assembly (100). The pivoting piece (20) comprises compression springs (21), located at the middle portion of the floor frame assembly (100). The anterior (front) portion of the floor frame assembly comprises a sliding bolt latch (16) along with a square carabiner (17) to lock the T-bar and prevent it to come out from the floor frame assembly (100). The floor frame assembly (100) further comprises a second lock system at the posterior (end) portion with a quick release pin (15).

FIGS. 9A and 9B illustrate the side view of the damping system found inside the floor frame assembly. It comprises an angled rail (19) and a pivoting piece (20). The pivoting piece (20) is equipped with high-load compression springs (21) on one end that activates when the T-bar (12) reaches its final position, effectively and advantageously dampening vibrations and minimizing impact on the mobile medical radioisotope elution system during transit. FIG. 9A shows the pivoting piece at its lowest point when the elution or infusion system is not docked inside. FIG. 9B shows the pivoting piece in the raised position when the elution or infusion system is docked inside. The quick-release pin (15) is a high-capacity locking mechanism that secures one end of the T-bar (12) and/or elution or infusion system while maintaining its alignment, yet remains user-friendly for quick installation and removal. On the opposite end, the sliding bolt latch (16) provides a secondary locking point, advantageously preventing any unintended movement of the T-bar (12) or the elution or infusion system. To further secure the sliding bolt latch (16), a square carabiner (17) is used by locking it firmly in place and ensuring the mobile medical radioisotope elution system remains stable and secure under all conditions during transportation.

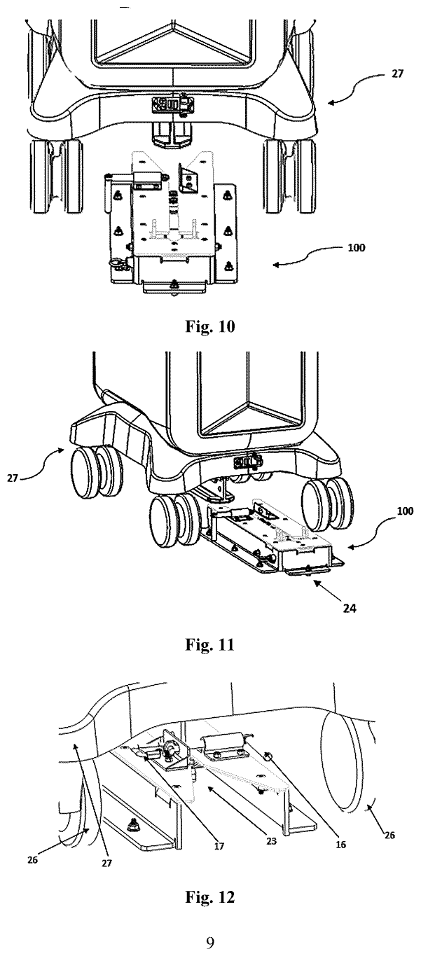

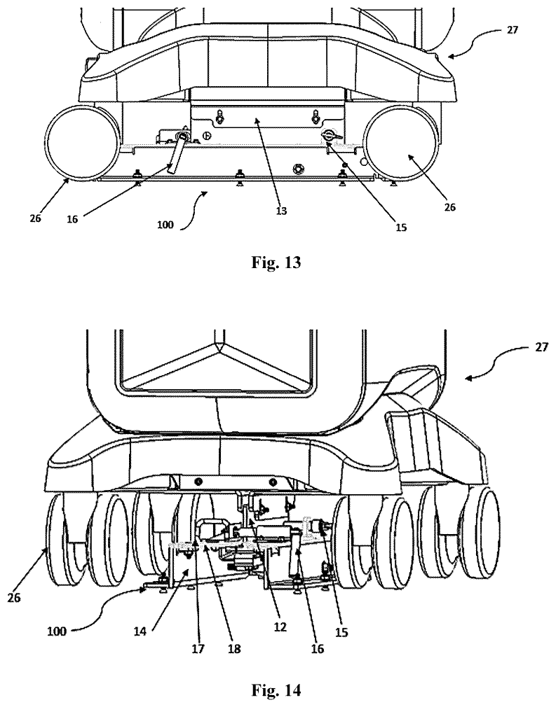

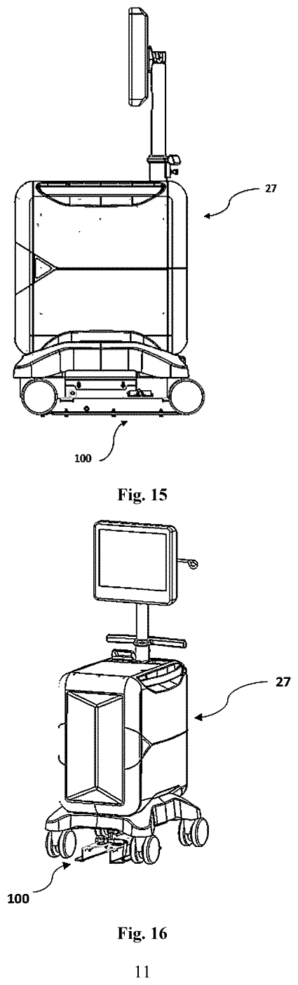

FIG. 10 illustrates the initial position or alignment of the floor frame assembly (100) and the elution or infusion system before docking with each other. FIGS. 11-16 show the fittings and docketing of the elution or infusion system along with the wheels (26) inside the floor frame assembly (100) from different positions.

We claim:

1. A mobile adaptation kit, comprising:
a) a floor frame assembly configured to be installed in a transport vehicle configured to support a mobile medical radioisotope elution system; and
b) a T-bar configured to be slid into and out of a slot in the floor frame assembly,
wherein the T-bar comprises a gasket and one or more polymer sheets;
wherein the floor frame assembly comprises:
i) a floor frame configured to be attached to the transport vehicle floor;
ii) a floor frame cover with a top portion that defines a slot to install the T-bar into and out of the floor frame assembly;
iii) an angled rail on each side of the floor frame assembly that installs under the floor frame cover;
iv) a pivoting piece at the centre of the floor frame assembly;
v) a quick-release pin with a high-capacity pin configured to lock one end of the T-bar in place with the floor frame; and
vi) a locking mechanism that locks at one end of the T-Bar in position, in order to prevent sliding out of the radioisotope elution system;
wherein the gasket is configured to absorb vibration during transportation; and the one or more polymer sheets are configured to reduce friction when inserting into the floor frame assembly.

2. A transport vehicle comprising a medical imaging system and a medical radioisotope system, wherein the medical radioisotope system is mounted to a floor surface of the transport vehicle using a mobile adaptation kit, wherein the mobile adaptation kit comprises:
a) a floor frame assembly installed in a floor surface of the transport vehicle configured to support the mobile medical radioisotope system; and
b) a T-bar attached to the mobile medical radioisotope system and configured to slide into and out of the floor frame assembly,
wherein the T-bar comprises a gasket and one or more polymer sheets;
wherein the floor frame assembly comprises:
i) a floor frame that is attached to the transport vehicle floor;
ii) a floor frame cover with a top portion that defines the slot to pass the T-bar into and out of the floor frame assembly to install the T-bar in the floor frame cover;
iii) an angled rail on each side of the floor frame assembly that installs under the floor frame cover;
iv) a pivoting piece at the centre of the floor frame assembly, installed with one or more high load compression springs;
v) a quick-release pin with a high-capacity pin that locks one end of the T-bar in place with the floor frame; and
vi) a locking mechanism that locks at one end of the T-Bar in position, in order to prevent sliding out of the radioisotope elution system;
wherein the gasket is configured to absorb vibration during transportation; and the polymer sheets are configured to reduce friction when inserting the T-bar into the floor frame assembly;
wherein the said mobile adaptation kit securely holds the medical radioisotope system to the floor of the transport vehicle and is configured to provide protection from damage to the medical radioisotope system during transportation;
wherein the transport vehicle further comprises:
a) at least one cubicle within the vehicle, dimensioned to accommodate a medical imaging apparatus;
b) an inertial measurement unit (IMU) for detecting and monitoring vehicle orientation and motion; and accurately and objectively monitoring of patient movement;
c) a power supply system comprising an on-board generator and battery backup configured to supply continuous power to medical imaging and diagnostic equipment; and
d) a sensor system; wherein the sensor system is selected from one or more sensors selected from the group consisting of temperature sensor, pressure sensor, motion sensor, vibration sensor, current sensor, speed sensor, air detection sensor, occlusion detection sensor, humidity sensor, position sensor, a wind sensor, a wave sensor, an engine control sensor, an electromagnetic field sensor, and voltage sensor or combination thereof.

3. A mobile adaptation kit, comprising:
a) a floor frame assembly installed in a transport vehicle configured to support a mobile medical radioisotope elution system; and
b) a T-bar that slides into and out of the floor frame assembly, wherein the T-bar comprises a gasket and nylon sheets; and wherein the gasket is configured to absorb vibration during transportation; and the nylon sheets are configured to reduce friction when inserting into the floor frame assembly;
wherein the floor frame assembly comprises:
i) a floor frame that configured to be attached to the transport vehicle floor;
ii) a floor frame cover with a top portion that creates the slot to install the T-bar into and out of the floor frame assembly;
iii) an angled rail positioned on each side of the floor frame assembly that installs under the floor frame cover, wherein the angled rail is used as a guide to align the T-bar into position;
iv) a pivoting piece at the centre of the floor frame assembly, installed with one or more high load compression springs, wherein the pivoting piece is activated when the T-bar is in the final position;
v) a quick-release pin with high-capacity pin that locks one end of the T-bar in place with the floor frame;
vi) a sliding bolt latch that locks other end of the T-Bar in position, in order to prevent sliding out of the radioisotope elution system; and
vii) a square carabiner that locks the sliding bolt latch in place;
wherein the floor frame cover holds a quick-release pin and a sliding bolt latch;
wherein the said mobile adaptation kit securely holds the medical radioisotope elution system to the floor of a vehicle and ensures protection from undesirable damage to the medical radioisotope elution system during transportation; and
wherein the mobile adaptation kit further comprises a sensor system; wherein the sensor system is selected from one or more sensors selected from the group consisting a temperature sensor, pressure sensor, motion sensor, vibration sensor, self-levelling sensor, current sensor, speed sensor, power supply sensor, air detection sensor, occlusion detection sensor, humidity sensor, position sensor, a wind sensor, a wave sensor, and voltage sensor or combination thereof.

4. The mobile adaptation kit according to claim 1, wherein the mobile adaptation kit further comprises one or more torx screws, and one or more ball bearings for a shaft.

5. The mobile adaptation kit according to claim 1, wherein the mobile medical radioisotope elution system comprises a strontium-rubidium elution system.

6. The mobile adaptation kit according to claim 1, wherein the pivoting piece further comprises a high load fastener mount compression spring.

7. The mobile adaptation kit according to claim 1, wherein the pivoting piece comprises a pivoting shaft and a flange locknut.

8. The mobile adaptation kit according to claim 1, wherein the locking mechanism comprises a sliding bolt latch; and a square carabiner that locks the sliding bolt latch in place; wherein in use the sliding bolt latch is used to prevent sliding out of the radioisotope elution system; and wherein the sliding bolt latch locks other end of the T-Bar in position to prevent sliding out of the radioisotope elution system.

9. The mobile adaptation kit according to claim 1, wherein the angled rail is configured to align the T-bar into position.

10. The mobile adaptation kit according to claim 1, wherein the pivoting piece is activated when the T-bar is in the final position.

11. The mobile adaptation kit according to claim 6, wherein the high load fastener mount compression springs are configured to dampen vibration during transit and minimize the vibration impact on a radioisotope elution system.

12. The mobile adaptation kit according to claim 6, wherein the high load fastener mount compression spring is selected to hold a weight from about 100 kg to about 800 kg.

13. The mobile adaptation kit according to claim 1, wherein the adaptation kit further comprises side plates that attach to the radioisotope elution system.

14. The mobile adaptation kit according to claim 1, wherein the adaptation kit further comprises socket head screw, one or more screws, and one or more washers.

15. The mobile adaptation kit according to claim 1, wherein the one or more polymer sheets comprise a polymer selected from one or more of polypropylene, nylon, polyethylene, polytetrafluoroethylene (PTFE), polyethylene terephthalate (PET), poly-ether-ether-ketone (PEEK), and Acetal.

16. The mobile adaptation kit according to claim 1, wherein the locking mechanism further comprises a latch strike-plate; a latch housing; a latch assembly; nylon-insert locknut; and flat head torx screw; and wherein the latch assembly comprises a latch body; a latch handle; and a socket head screw.

17. The mobile adaptation kit according to claim 1, wherein the mobile adaptation kit secures the generator, computer, waste bottle, and other critical components throughout the transportation process.

18. The mobile adaptation kit according to claim 1, wherein the mobile adaptation kit further comprises a sensor system; and wherein the sensor system comprises at least one or more sensors selected from a group consisting of a temperature sensor, pressure sensor, motion sensor, vibration sensor, current sensor, speed sensor, air detection sensor, occlusion detection sensor, humidity sensor, position sensor, a wind sensor, a wave sensor, an engine control sensor, an electromagnetic field sensor, and voltage sensor.

19. The mobile adaptation kit according to claim 1, wherein the mobile adaptation kit is attached at the floor of a mobile medical radioisotope transport system or medical vehicle, wherein the mobile medical radioisotope transport system or medical vehicle comprising:
a vehicle structure configured to house one or more medical imaging systems; and
at least one cubicle within the vehicle, dimensioned to accommodate a medical imaging modality;
wherein the medical imaging system is selected from the group consisting of PET, PET/CT, dynamic PET, SPECT, SPECT/CT, MRI, CT, ultrasound-CT, PET/MRI and combination thereof;
wherein the cubicle is defined by physical walls or by a virtual boundary within the internal volume of the vehicle; and
wherein the cubicle includes additional space adjacent to the medical imaging system to facilitate operator access or patient positioning.

20. The mobile adaptation kit according to claim 19, wherein the mobile medical radioisotope transport system or transport vehicle comprises a method for providing mobile medical imaging services, comprising:
transporting a vehicle equipped with at least one medical imaging modality to a predetermined or designated location for diagnostic service delivery;
stabilizing the vehicle at the location using an integrated sensor system configured to assess terrain and adjust vehicle positioning for optimal operational stability;
activating an onboard power supply subsystem, comprising a generator and battery backup, to initiate and sustain operation of the medical imaging modality selected from the group consisting of PET, PET/CT, dynamic PET, SPECT, SPECT/CT, MRI, CT, ultrasound-CT, PET/MRI and the like;
initiating the vehicle sensor system vehicle's sensor system to monitor environmental and operational parameters during imaging procedures;
registering a patient through a digital interface configured to capture patient identification, medical history, and consent documentation;
performing a diagnostic imaging procedure using the imaging modality selected from the group consisting of PET, PET/CT, dynamic PET, SPECT, SPECT/CT, MRI, CT, ultrasound-CT, PET/MRI and the like;
transmitting the acquired imaging data to a remote diagnostic center via a secure data transmission subsystem for expert analysis and interpretation; and
receiving and storing diagnostic results within the onboard data management system for subsequent clinical use, patient consultation, or integration into electronic health records.
